(19) United States
(12) Patent Application Publication
(10) Pub. No.: US 2025/0284383 A1
(43) Pub. Date: Sep. 11, 2025

(54) Multi-axis swipe or drag-based response format for psychometric assessments

(71) Applicant: Michael James Russiello, Gainesville, VA (US)

(72) Inventor: Michael James Russiello, Gainesville, VA (US)

(21) Appl. No.: 18/597,769

(22) Filed: Mar. 6, 2024

(51) Int. Cl. G06F 3/04845; G06F 3/04817; G06F 3/04842; G06F 3/0485; G06F 3/0486; G09B 7/06

(52) U.S. Cl. CPC G06F 3/04845; G06F 3/04817; G06F 3/04842; G06F 3/0485; G06F 3/0486; G09B 7/06

(57) ABSTRACT

A computer-implemented method of gathering responses to questions and statements administered as part of a psychometric assessment, such that it allows the participant to swipe or drag a rendered graphic object that can include an image, text, and or other media assets, or any combination of image and text across the screen in permitted directions along two or more axes in order to register a specific response to the presented question or statement. The method includes presenting helpful arrow icons and anchor text to assist the test-taker in choosing the proper direction and axis.

### CROSS-REFERENCE TO RELATED APPLICATIONS

Provisional Patent Application No. 61/698,277, Filed Mar. 7, 2023, Title of invention: A multi-axis swipe or drag-based response format for psychometric assessments.

### PRIOR ART

### U.S. Patent Documents

U.S. Pat. No. 5,671,409 September 1997 Fatseas et al.
D555663 S November 2007 Nagata et al.
D568892 May 2008 Stabb et al.
U.S. Pat. No. 8,086,558 B2 December 2011 Dewar
D755814 S May 2016 Rad et al.
D779540 S February 2017 Rad et al.
D780775 S March 2017 Rad et al.
D781311 S March 2017 Rad et al.
U.S. Pat. No. 10,503,346 B2 December 2019 Havilio

### Other Publications

Understanding Swipe-Based vs. Algorithm-Based Dating Apps Jan. 29, 2023 https://www.makeuseof.com/swipe-based-vs-algorithm-based-dating-apps/
The Psychology of Swiping in Apps Sep. 6, 2018 App Partner Academy https://medium.com/app-partner-academy/the-psychology-of-swiping-in-apps-464895b2b485
Slider Scale or Text Box: How Response Format Shapes Responses Journal of Consumer Research, Volume 45, Issue 6, April 2019, Pages 1274-1293, https://doi.org/10.1093/jcr/ucy057
Swipe Worth the Hype? Masters Thesis June 2022, Tilburg University, Tilburg http://arno.uvt.nl/show.cgi?fid=159034
Swipe right on personality: a mobile response latency measure Nathan W. Weidner, Richard N. Landers, Journal of Managerial Psychology, ISSN: 0268-3946, Feb. 18, 2020

### BACKGROUND OF THE INVENTION

### Technical Field

The present disclosure relates to presenting content objects for self-ratings to individuals as one or more inputs to a psychometric assessment that evaluates one or more traits or characteristics of that individual.

### Field of the Invention

CPC G06F 3/048, G06F 3/0482, G06F 3/04842, G06F 3/04817, G06F 3/0485, G06F 3/0486

Description of Related Art: Referenced patents include swipe or drag-based interaction formats used on computing devices for various business applications such as online dating. Referenced papers include swipe or drag-based interaction formats psychometric self-report or ‘rating’ assessments. However, use is restricted to dichotomous response formats which employ two allowed directions on a single axis only, rather than multiple axes. Research presented in the papers shows that use of these dichotomous items requires significantly more items than a conventional Likert response format with more than two choice options. Adding additional items to a psychometric test places a higher burden on the test taker since they have more work to do in order to complete the test.

Background: Employment assessments assist employers in making critical decisions regarding their human capital. Computer and networking technology has lowered the cost of administering an employment testing program and increased their use significantly. Beyond cost reduction, advances in information technology offer opportunities to improve the reliability of psychometric assessments, as well as reduce the test taker burden through new response formats.

Swipe or drag-based formats can be applied to online psychometric assessments. Instead of selecting a choice radio button on a screen, a test taker can swipe (or drag if they are using a mouse) in two directions along a single axis to indicate his or her response to a question (item). The previously embodied swipe and drag-based implementations have been dichotomous. That is, there are only two directions along a single, usually horizontal, axis. Typically, these two allowed responses represent the two extremes of the scale in use. For instance, a left swipe could represent a response of “Not like me” and a right wipe might indicate a response of “Like me.” This approach does not allow test-takers to indicate other gradations along the rating scale, such as “Somewhat like me” or “Mostly like me.”

At least one study has demonstrated that test-takers enjoy utilizing swipe or drag-based formats at least as much or more than traditional radio button formats on a question for question basis. However, since the items are dichotomous, more items are required to obtain an acceptable level of test reliability. The extra items add to the test taker burden due to the longer time required to complete the assessment. The large number of items currently embodied in many existing psychometric assessments (100 or more) can lead to test-taker boredom and loss of concentration. Adding additional items to compensate for the use of dichotomous items exacerbates this problem and makes any dichotomous format un-usable in many applications.

Another problem is the test-taker burden per item. Completing a test typically places a cognitive load on the candidate. The more difficult the question, the higher the burden. Test-takers can find it more difficult to make a decision allowing for only one extreme or the other, rather than one that allows them to make a choice that is in between, making dichotomous items less useful in psychometric assessments.

Another problem with static items is that they quickly become boring and monotonous, particularly if they utilize text only and do not include any accompanying graphics. Test-takers can easily lose focus and the quality of their responses can decrease.

### SUMMARY OF THE INVENTION

The invention adds one or more additional axes to single axis swipe or drag based items, enabling any number of possible responses along a rating scale. By enabling any number of possible responses, the invention avoids the need to add additional items to satisfy reliability concerns when using a swipe or drag-based response format in psychometric assessments. Additionally illustrative graphical elements can be combined with swipe-able or drag-able text to make the easier to understand and more visually interesting to the test-taker, which reduces the test-taker burden. Clickable or inert arrows and anchors can also be included to show the allowed directions and their meanings, and to allow the test-taker to simply choose a direction rather than swipe if they are made clickable, further reducing test-taker burden.

In one embodiment, a psychometric assessment uses swipe or drag-based questions (items) to collect ratings from test-takers. Each item allows for swiping or dragging along 5 different directions using 4 axes, as well as for clicking a button to select each allowed direction. Test-takers completing the assessment on computers or devices without a touch screen can use a mouse or other pointing device to drag or click. Test-takers using a mobile or touch screen-enabled device can use their finger or other pointing device, such as a stylus, to swipe or click in the desired direction. Test-takers can be allowed to go back one item in case they swiped or dragged incorrectly.

Each item is comprised of a text-based statement which is to be rated, which can be accompanied by images or video to further illustrate the meaning of the statement. In the current embodiment, this statement is accompanied by an image that illustrates the concept or trait represented by the statement. Both the text and the image are swiped or dragged as a single object. Other media, such as video can be used within the swipe-able object when appropriate.

Guiding arrow icons may be presented to instruct test-takers on the available directions.

To avoid spurious or inadvertent responses, the system requires that dragging or swiping be accomplished above a threshold speed. Multiple items are asked for each trait or characteristic being measured. Items can be sorted by trait or randomized. Items are then administered in sequence until all required items have received responses. Response information is them passed to a scoring and report generation system.

Any number of directions and axes are possible, including an infinite number. In another embodiments, the user can swipe or drag anywhere within a 180-degree arc to enter an infinite number of possible responses along the rating scale. In another embodiment, the user can swipe or drag anywhere within a 360-degree arc to enter an infinite number of possible responses along the rating scale. These embodiments allow the test-taker to select a response anywhere along a linear scale.

### BRIEF DESCRIPTION OF DRAWINGS

FIG. 1 is a diagram that shows a complete system operating in accordance with one embodiment of the invention.

FIG. 2 is a flowchart that shows a typical process flow for a system administering swipe or drag-based items as part of psychometric assessment, in accordance with one embodiment of the invention.

FIG. 3 shows one embodiment of an instructions screen preceding any practice or live swipe or drag-based items as part of a psychometric assessment.

FIG. 4 shows one embodiment of a swipe or drag-based item that allows for movement in 5 directions.

FIG. 5 shows the embodiment from FIG. 4 after a test-taker has swiped or dragged the movable components in the diagonal downward direction.

FIG. 6 depicts an embodiment with 5 possible directions using 4 axes.

FIG. 7 depicts an embodiment with 8 possible directions using 4 axes.

FIG. 8 depicts an embodiment with 9 possible directions using 5 axes.

### DETAILED DESCRIPTION OF THE INVENTION

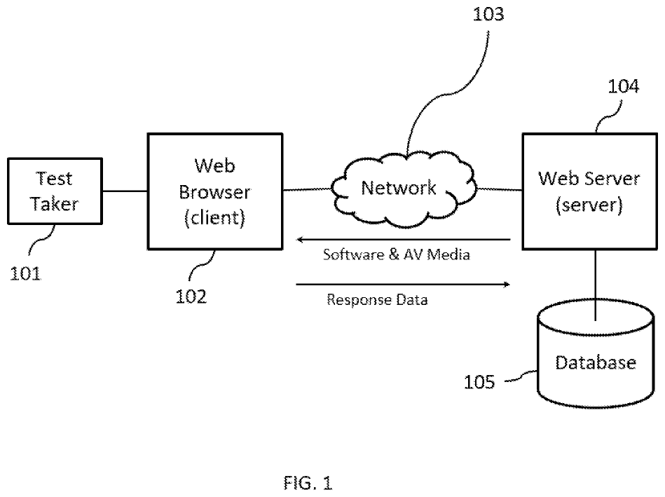

A system in accordance with one embodiment of the present invention is shown in FIG. 1. A test taker 101 interacts with a web browser 102 to access the assessment, which is delivered via the network 103 from a specialized server 104. The server provides client-side software such as HTML, javascript, and media assets that, working together, form individual items and the assessment within the web browser 102. The test taker responds to a series of prompts in order to advance through and complete the assessment. As the test taker responds, the response data is captured and sent via the network 103 to the server 104, which stores it within a database 105.

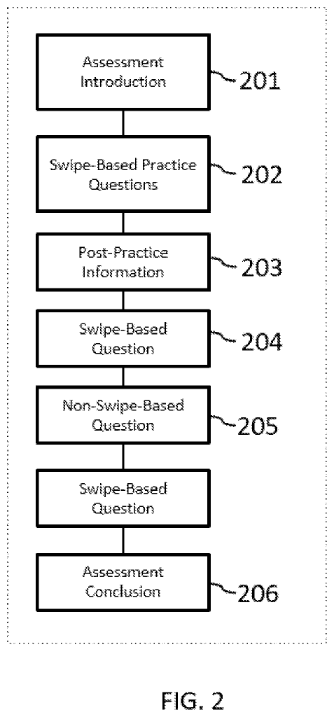

A process flowchart for one embodiment of an assessment that uses swipe or drag-based items is shown in FIG. 2. The test taker first views one or more introductory screens 201, then completes one or more practice questions 202 followed by a post-practice launch screen 203 that informs them they are about to begin the recorded questions 204 and 205. The recorded questions can use either swipe-based 204 or non-swipe-based 205 response formats. The assessment terminates with a screen acknowledging completion for the participant 206.

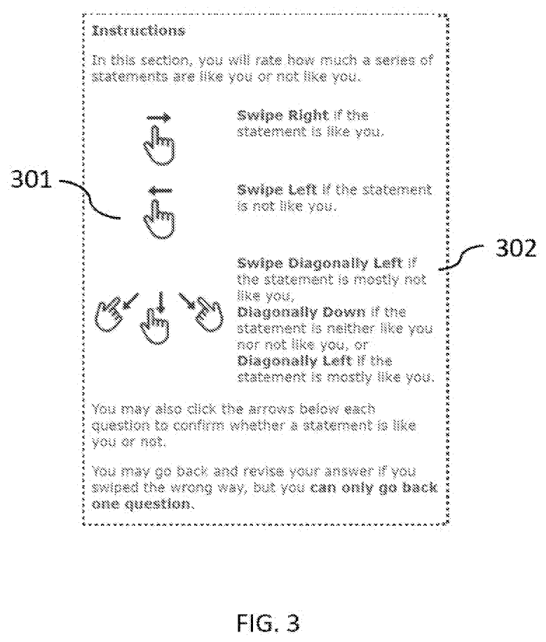

FIG. 3 depicts one embodiment of an instructions screen explaining how the swipe or drag item format works. Hand icons with arrows 301 and their accompanying text 302 indicate the meaning of swiping in a specific direction.

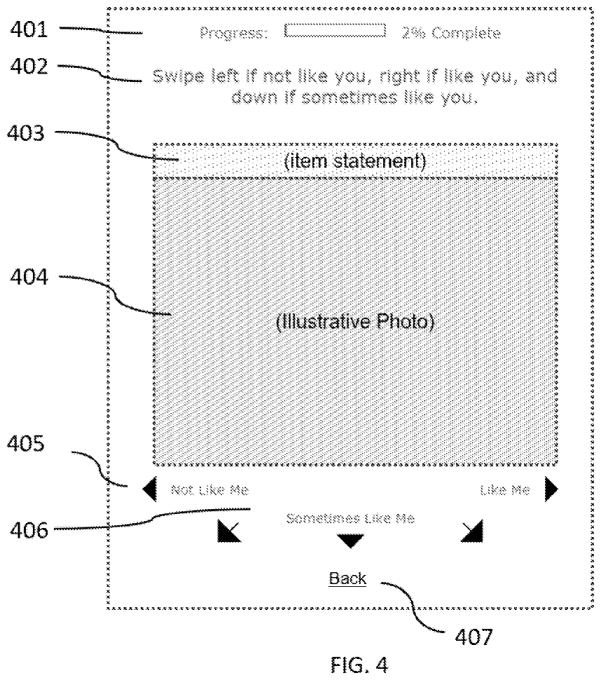

FIG. 4 shows one embodiment of a swipe or drag-based item using 5 possible directions using 4 axes. Allowed directions are Left, Down-Left, Down, Down-Right, and Right. Other embodiments could add additional axes and directions, including an infinite number of axes. Progress within the assessment is presented 401. Instructions are repeated 402. A swipe or drag-able graphic object consisting of text 403 and/or an image 404 is presented. Allowed directions are reinforced by image buttons 405 that can also be clicked to simulate the swipe or drag action. Text anchors highlighting the meaning of each direction are included 406. A ‘Back’ link 407 is provided in the case of inadvertent swipes or responses that a test-taker would like to change. Note that other than the swipe or drag-able graphic represented by 403 and or 404, all other components are optional for other embodiments. In this embodiment the clickable arrow buttons 405 and anchors 406 will move with the graphic 403 and 404 but they can be static in other embodiments. Additionally, the number of allowed directions, ranging from 3 to infinite, each representing a position along the rating scale can be allowed for movement in other embodiments. The broken line showing the borders of the display screen is for the purpose of illustrating portions of the article and forms no part of any claim.

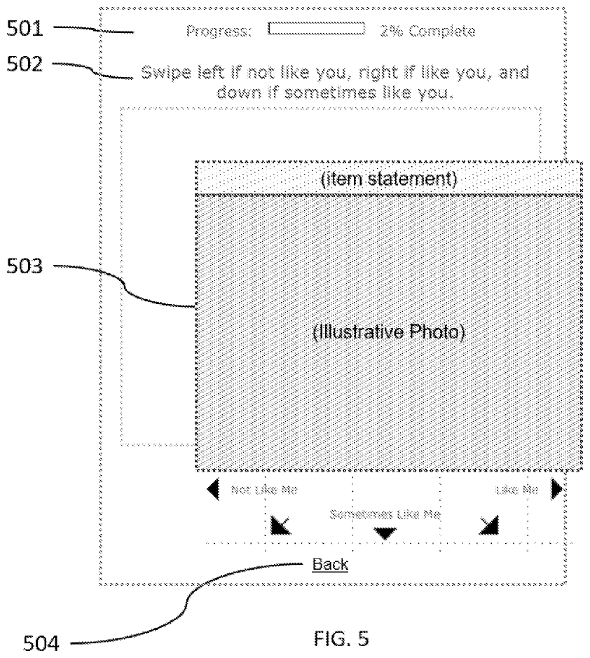

FIG. 5 depicts the same interface as FIG. 4 except the test-taker has moved the movable components in a permitted direction along a permitted axis by swiping with his or her finger or dragging. The progress within the assessment is displayed 501. Instructions are included to prompt the test-taker 502. The movable components 503 move as a group and only in a permitted direction along a permitted axis.

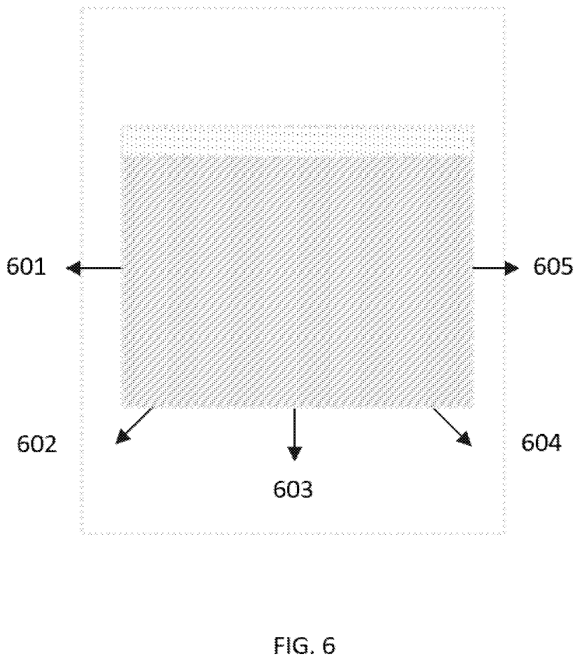

FIG. 6 depicts an embodiment of the interface with 5 possible directions using 4 axes. Test takers are permitted to swipe or drag either left 601, down-left 602, down 603 down-right 605 or right 605. Such an embodiment could be a practical replacement for a traditional radio button item with five radio buttons.

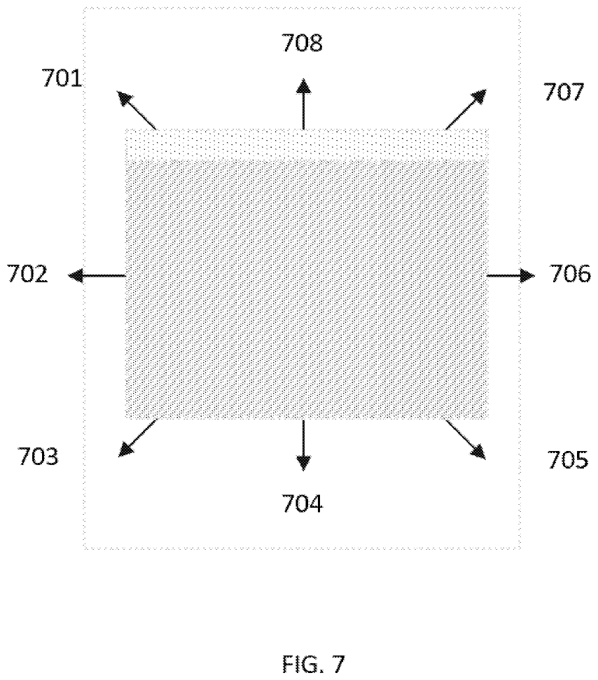

FIG. 7 depicts an embodiment of the interface with 8 possible directions using 4 axes. Test takers are permitted to swipe or drag either up-left 701, left 702, down-left 703, down 704, down-right 705, right 706, up-right 707 or up 708. Such an embodiment could be a practical replacement for a traditional radio button item with 8 radio buttons.

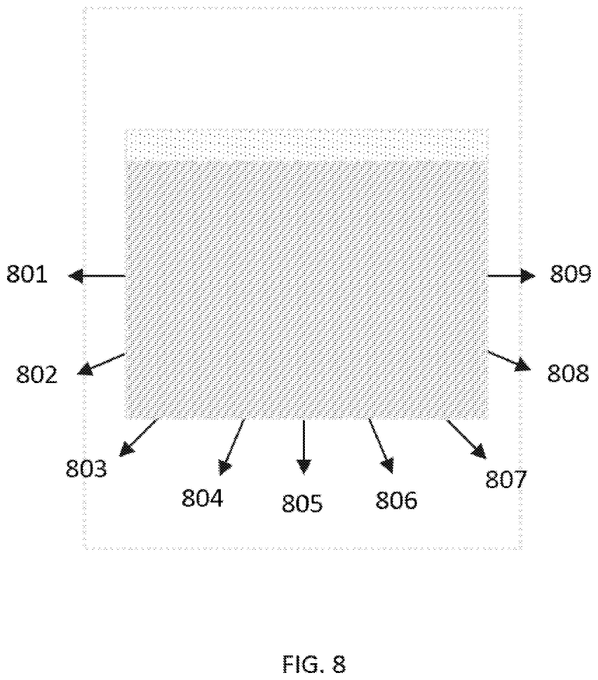

FIG. 8 depicts an embodiment of the interface with 9 possible directions using 7 axes. Test takers are permitted to swipe or drag either left 801, left-left-down 802, down-left 803, down-down-left 804, down 805, down-down-right 806, down-right 807, right-down 808, or right 809. Any number of directions and axes may be used to support the response range desired in different embodiments. Such an embodiment could be a practical replacement for a radio button item with 9 radio buttons. Additional axes and scales can be added to achieve the desired number of response options. Additionally, an infinite number of axes can be supported, which enables a continuous rating scale.

1. A computer-implemented, including mobile devices, method of enabling participants in employment assessments to indicate their response to a question or statement by swiping or dragging a graphic object rendered on the screen and containing any combination of text, video, shaded areas, or images, in either one or two directions along two or more axes, with each combination of axis and direction representing a different response.

2. The method of claim 1, wherein any number of allowed axes, with either one or both directions per axis can be incorporated to support any number of responses.

3. The method of claim 1, wherein the swipe or drag can be accomplished by the participant with their finger, stylus, mouse, or other pointing device depending on the computer and capabilities of the monitor or screen in use.

4. The method of claim 1, wherein the rendered graphic to be swiped or dragged includes directional buttons or icons that indicate the allowed directions and axes for swiping or dragging and may also offer an alternative response modality by acting as clickable buttons, icons or text links representing some or all of the allowed response directions and where a click on such a button is equivalent to a swipe in the indicated direction and axis.

5. The method of claim 1, wherein a ‘back’ button is included to allow a participant to replace a previous response with a new response.

6. The method of claim 1, wherein the system monitors the speed of swiping or dragging against a minimum required speed in order to determine if the swipe is acceptable or not, accelerating the graphic off the screen is it acceptable or returning it the its original position if is not acceptable once the swipe or dragging gesture is completed.

7. The method of claim 1, wherein the system monitors the direction of swiping against the allowed axes or allowed rang e of directions within pre-defined tolerances, and restricts the movement of the graphic to the different allowed directions.

8. The method of claim 1, wherein the system monitors the distance, speed, and axis of swiping against pre-defined tolerances to determine whether the swipe indicates a valid response, and then automatically finishes the swipe by moving the rendered graphic off the screen without further interaction from the participant if the response is valid, or back to the original position if the swipe is not valid when the swiping or dragging gesture is completed by the test-taker.

9. The method of claim 1, wherein participants can go back to previous questions using an image or text link to revise their swipe or drag gesture, in order to review or change their answer.

10. The method of claim 1, wherein the system saves valid responses in either an ephemeral or persistent storage medium for either real-time or post processing, and/or for determining the next question to present, and advances to the next question in the assessment by removing the currently rendered graphic and replacing it with a new graphic representing a new item (question).
